(19) United States
(12) Patent Application Publication
(10) Pub. No.: US 2006/0165638 A1
(43) Pub. Date: Jul. 27, 2006

(54) Compositions comprising at least one copolymer (a) and at least one copolymer (b) and use thereof in cosmetic preparations

(75) Inventors: Izaskun Manteca Zuazo, Ludwigshafen (DE); Marcos Gomez, Hidelberg (DE); Son Nguyen-Kim, Hemsbach (DE)

Correspondence Address: CONNOLLY BOVE LODGE & HUTZ LLP, SUITE 800, 1990 M STREET NW, WASHINGTON, DC, 20036-3425, US

(73) Assignee: BASF AKTIENGESELLSCHAFT, Ludwigshafen (DE)

(21) Appl. No.: 10/534,229

(22) PCT Filed: Nov. 17, 2003
(86) PCT No.: PCT/EP03/12816

§ 371 Date: May 6, 2005

(30) Foreign Application Priority Data
Nov. 29, 2002 (DE) 102 56 148.6

(51) Int. Cl. A61K 8/81

(52) U.S. Cl. 424/70.15; 424/70.16

(57) ABSTRACT

Disclosed is a composition comprising (A) copolymer made up of (A-1) at least one ethylenically unsaturated, radically copolymerizable monomer of general formula Y—NR¹—C(V)—NHR², wherein the substituents have the following meaning: Y represents a radically polymerizable ethylenically unsaturated radical; V represents O, S, or NH; R¹, R² independently represent H or a C₁ to C₈ alkyl group or jointly represent a bridging C₂ to C₄ alkylene group that can be substituted up to twice with a C₁ to C₄ alkoxy group and/or hydroxyl group; (A-2) at least one unsaturated monomer of general formula X—C(O)CR⁷═CHR⁶, wherein the substituents have the following meaning: X is selected among the group of radicals —OH, —OR⁸, NH₂, —NHR⁸, N(R⁸)₂, radicals R⁸ being identically or differently selected among the group comprising —H, C₁-C₄₀ linear-chain or branched-chain alkyl radicals, N,N-dimethylaminoethyl, 2-hydroxyethyl, 2-methoxyethyl, 2-ethoxyethyl, hydroxypropyl, methoxypropyl, or ethoxypropyl; R⁷ and R⁶ are independently selected among the group comprising —H, C₁-C₈ linear-chain or branched-chain alkyl chains, methoxy, ethoxy, 2-hydroxyethoxy, 2-methoxyethoxy, and 2-ethoxyethyl, (B) at least one additional copolymer that is different from (A) and is made up of (B-1) at least one monomer of general formula (III), wherein R⁹ represents H, alkyl with 1 to 8 C atoms, R¹⁰ represents H, methyl, R¹¹ represents alkylene with 1 to 24 C atoms, which is optionally substituted by C₁-C₆ alkyl, R¹², R¹³ represent a C₁-C₄₀ alkyl radical, Z represents nitrogen if x=1 or oxygen if x=0, and (B-2) at least one ethylenically unsaturated monomer.

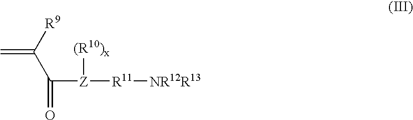

The invention relates to compositions comprising at least one copolymer (A) and at least one copolymer (B), and to the use thereof in cosmetic preparations.

For cosmetic preparations, in particular those based on oil, cream or lotion, properties such as good feel on the skin, water-binding capacity and absorption, consistency and ability to soak in are decisive parameters. These properties are influenced by the physical behavior of the cosmetic preparations under the action of mechanical forces. The rheological profile of a cosmetic preparation is of great importance for said properties.

To influence the rheological profile, water-insoluble polymeric thickeners are, for example, added to cosmetic preparations. Examples of known thickening additives are fatty alcohol ethoxylates, fatty acid alkylolamides, ethoxylated glucose esters, ethoxylated partial glycerides or sodium chloride. Too much sodium chloride can, however, have the opposite effect in a formulation based on fatty alcohol ether sulfate, and the product becomes less and less viscous, resulting in extreme cases in phase separation.

Another way of thickening consists in utilizing the viscosity-increasing effect of surfactant mixtures. Alkylbetaines and alkyl polyglycosides thicken, in small additions, formulations based on fatty alcohol ether sulfate.

Pseudoplastic products with gel character, which are suitable, for example, for application from the tube, are obtained by adding polymeric gel formers, e.g. polyacrylic acid, xanthan or hydroxypropylmethylcellulose.

EP 0.824 914 B1 describes a thickened bodycare composition which comprises a polymeric rheology modifier. This is obtainable by polymerization of C₁-C₆-alkyl (meth)acrylic esters, (meth)acrylamides and so-called associative monomers.

WO 97/36572 describes a cosmetic preparation which comprises at least one silicone oil and a thickener, where the thickener is a polymer which contains silicone groups and, as further functional groups, for example urea groups.

It is an object of the present invention to provide novel polymers which are suitable as thickeners, in particular as thickeners in cosmetic preparations. Of particular interest were cosmetic preparations which comprise an oil phase.

DE 34 43 964 A1 and DE 39 02 103 A1, describe binders for aqueous gloss paints which consist of multiphase emulsion polymers. These are composed of a hard core material and a soft shell material. Monomers with ureido groups may be involved in the composition of the shell material.

DE 39 02 555 A1 describes aqueous polyacrylate systems for the coating of plastic surfaces which can comprise up to 10% by weight of a polymerizable ureido compound, such as, for example, N-methacrylamidomethyleneurea, N-(2-methacryloyloxyethyl)ethyleneurea, and N-vinylimidazole.

DE 39 02 067 A1 describes film-forming, self-crosslinking aqueous plastics dispersions comprising an emulsion polymer which may contain a free-radically polymerizable monomer with a ureido group. These dispersions are used for painting plastic surfaces.

DE 43 34 178 A1 (=EP 722 477 A1, WO 95/09896 A1) describes aqueous polymer preparations which comprise a polymer which has ureido groups, and a polyaldehyde compound. These preparations are used to produce coatings with increased adhesion to the substrate and also with increased internal strength.

DE 100 41 680 A1 describes aqueous polymer dispersions which may comprise urea derivatives, such as, for example, N-(2-methacryloxyethyl)ethyleneurea or N-(2-methacryloxyethyl)thiourea up to 5% by weight as monomers. These dispersions are suitable as binders in coating compositions and ensure high gloss and high blocking resistance of the coatings.

U.S. Pat. No. 3,645,965 describes synthetic polymers which, as a result of the incorporation of imidazolidin-4-ones, have increased light and temperature stability.

U.S. Pat. No. 3,876,657 describes the preparation of 1-substituted imidazolidin-2-ones and the use of these compounds as monomers for polymers and copolymers.

U.S. Pat. No. 5,852,123 describes graft polymers comprising a urea or imide group and the use of these graft polymers for dispersing pigments. The polymers consist of 2 to 97% by weight of a backbone of a polymer of ethylenically unsaturated monomers, 97 to 2% by weight of a macromer which is grafted via a terminal, ethylenically unsaturated group to the backbone, and at least 1% by weight of a dispersing agent which contains an imide group and/or a urea group.

U.S. Pat. No. 5,972,431 describes aqueous preparations for coating compositions, inks and agricultural compositions which comprise a cyclic urea derivative to reduce the surface tension.

EP 0 124 713 B1 describes imidazolidinone monomers and also polymers of these monomers and ethylenically unsaturated monomers, and also the use of these polymers as formaldehyde-free binders for nonwoven textiles.

EP 0 609 793 A2 describes an aqueous binder comprising a film-forming aqueous polymer dispersion and a crosslinking component. The polymer dispersion is an emulsion polymer with polymerized units of an ethylenically unsaturated, free-radically polymerizable monomer with a lateral alkyleneurea group. The crosslinking component consists of polyfunctional aldehydes.

EP 1 000 610 A1 (DE 19850363) describes the use of polymers to which derivatives of ethylene urea are bonded in cosmetics and as hair-setting polymers. These polymers are characterized by improved wash-off with reduced solution viscosity and unchanged setting action.

The present invention provides compositions comprising

(A) copolymer of
(A-1) at least one ethylenically unsaturated, free-radically copolymerizable monomer of the formula (I)
   Y—NR¹—C(V)—NHR² (I),
   where the substituents have the following meanings:
   Y=an ethylenically unsaturated radical capable of free-radical polymerization
   V=O, S or NH
   R¹, R²=independently of one another H or a C₁-C₈-alkyl group, or both together a bridging C₂-C₄-alkylene group which may be substituted up to twice by a C₁-C₄-alkoxy group and/or hydroxyl group,
(A-2) at least one unsaturated monomer of the formula (II)
   X—C(O)CR⁷═CHR⁸ (II),
   where the substituents have the following meanings:
   X is chosen from the group of radicals —OH, —OR⁸, NH₂, —NHR⁸, N(R⁸)₂;
   the radicals R⁸ may be identical or different and are chosen from the group consisting of —H, C₁-C₄₀ linear- or branched-chain alkyl radicals, N,N-dimethylaminoethyl, 2-hydroxyethyl, 2-methoxyethyl, 2-ethoxyethyl, hydroxypropyl, methoxypropyl or ethoxypropyl;
   R⁷ and R⁶ are independently of one another chosen from the group consisting of —H, C₁-C₈ linear- or branched-chain alkyl chains, methoxy, ethoxy, 2-hydroxyethoxy, 2-methoxyethoxy and 2-ethoxyethyl.

(B) at least one further copolymer different from (A) of
(B-1) at least one monomer of the formula (III) 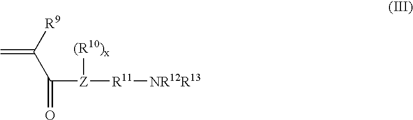
   where
   R⁹=H, alkyl having 1 to 8 carbon atoms,
   R¹⁰=H, methyl,
   R¹¹=alkylene having 1 to 24 carbon atoms, optionally substituted by C₁-C₆-alkyl,
   R¹², R¹³=C₁-C₄₀ alkyl radical,
   z=nitrogen when x=1 or oxygen when x=0. and
(B-2) at least one ethylenically unsaturated monomer.

Suitable as monomer (A-1) are, in particular, compounds where V=O, so-called ureido compounds.

Suitable as monomer (A-1) are compounds where V=S, so-called thioureido compounds.

Suitable as monomer (A-1) are compounds where V=NH, so-called guanidino compounds.

In a preferred embodiment, at least one compound of the formula (Ia) is used as monomer (A-1) 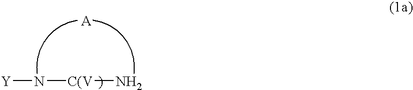
where A=a 2- or 3-membered alkylene group optionally having a carbonyl group.

Particular preference is given to compounds of the formula (Ia) in which A is an unsubstituted 2-membered alkylene group.

Particular preference is given to compounds of the formula (Ia) in which V=O.

In a preferred embodiment, R¹ and R² together form an optionally substituted bridging C2-alkylene group.

Particular preference is given to compounds of the formula (Ia) in which A is an unsubstituted 2-membered alkylene group and in which V=O.

In a preferred embodiment, at least one compound of the formula (Ib) is used as monomer (A-1) 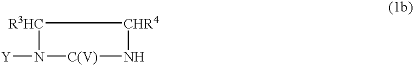
where R³ and R⁴, independently of one another, are H, —OH, —NH, C₁-C₈-alkyl.

Particular preference is given to compounds of the formula (Ib) in which V=O.

Particular preference is given to compounds of the formula (Ib) in which R₃ and R₄=H.

Particular preference is given to compounds of the formula (Ib) in which V=O, R₃ and R₄=H

and Y═CH₂═C(CH₃)—CO—O—(CH₂)₂—.

Particular preference is given to compounds of the formula (Ib) in which V=S, R₃ and R₄=H and Y═CH₂═C(CH₃)—CO—O—(CH₂)₂—.

Particular preference is given to compounds of the formula (Ib) in which V=NH, R₃ and R₄=H and Y═CH₂═C(CH₃)—CO—O—(CH₂)₂— (=ureido methacrylate).

As Y in formula (I), any ethylenically unsaturated radical capable of free-radical polymerization is suitable.

In a preferred embodiment of the present invention, the ethylenically unsaturated radical Y used is a radical of the following formula (IV)
Y═CH₂═CR⁵—CO—W—(CH₂)ₙ— (IV),
where
R⁵=H, CH₃
W=O, NH
n=2 to 8, in particular 2 to 4.
Examples of Y which may be mentioned:
CH₂═CH—CO—O—(CH₂)₂—; CH₂═CH—CO—NH—(CH₂)₂—; CH₂═C(CH₃)—CO—O—(CH₂)₂—; CH₂═C(CH₃)—CO—NH—(CH₂)₂;
CH₂═CH—CO—O—(CH₂)₃—; CH₂═CH—CO—NH—(CH₂—; CH₂═C(CH₃)—CO—O—(CH₂—; CH₂═C(CH₃)—CO—NH—(CH₂)₃—;
CH₂═CH—CO—O—(CH₂)₄—; CH₂═CH—CO—NH—(CH₂)₄—; CH₂═C(CH₃)—CO—O—(CH₂)₄—; CH₂═C(CH₃)—CO—NH—(CH₂)₄—.

Y is particularly preferably CH₂═C(CH₃)—CO—O—(CH₂)₂—.

R₁ and R₂, R₃, R₄, R₆, R₇, R₉ can, independently of one another, be hydrogen, branched or unbranched C₁-C₈-alkyl chains, preferably methyl, ethyl, n-propyl, 1-methylethyl, n-butyl, 1-methylpropyl-, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, n-heptyl, 2-ethylhexyl and n-octyl.

Suitable as monomer (A-2) are monomers of the formula (II)
X—C(O)CR⁷═CHR⁶ (II),
where the substituents have the following meanings:
X is chosen from the group of radicals —OH, —OR⁸, NH₂, —NHR⁸, N(R⁸)₂;
the radicals R⁸ may be identical or different and are chosen from the group consisting of —H, C₁-C₄₀ linear- or branched-chain alkyl radicals, N,N-dimethylaminoethyl, 2-hydroxyethyl, 2-methoxyethyl, 2-ethoxyethyl, hydroxypropyl, methoxypropyl or ethoxypropyl;
R⁷ and R⁶ are, independently of one another, chosen from the group consisting of —H, C₁-C₈ linear- or branched-chain alkyl chains, methoxy, ethoxy, 2-hydroxyethoxy, 2-methoxyethoxy and 2-ethoxyethyl.

Representative but nonlimiting examples of suitable monomers (A-2) are, for example, acrylic acid and esters and amides thereof.

The esters can be derived from C₁-C₄₀ linear, C₃-C₄₀ branched-chain or C₃-C₄₀ carbocyclic alcohols, from polyfunctional alcohols having 2 to about 8 hydroxyl groups, such as ethylene glycol, hexylene glycol, glycerol, and 1,2,6-hexanetriol, from aminoalcohols or from alcohol ethers, such as methoxyethanol and ethoxyethanol or polyethylene glycols.

Monomers (A-2) which can likewise be used are substituted acrylic acids and esters and amides thereof, where the substituents on the carbon atoms are in the two or three position of the acrylic acid, and, independently of one another, are chosen from the group consisting of C₁-C₄ alkyl, —CN, COOH, particularly preferably methacrylic acid, ethacrylic acid and 3-cyanoacrylic acid. These esters and amides of these substituted acrylic acids can be chosen as described above for the esters and amides of acrylic acid.

Particularly suitable monomers (A-2) are acrylic acid, methacrylic acid, ethylacrylic acid, methyl acrylate, ethyl acrylate, propyl acrylate, n-butyl acrylate, isobutyl acrylate, t-butyl acrylate, 2-ethylhexyl acrylate, decyl acrylate, methyl methacrylate, ethyl methacrylate, propyl methacrylate, n-butyl methacrylate, isobutyl methacrylate, t-butyl methacrylate, 2-ethylhexyl methacrylate, decyl methacrylate, methyl ethacrylate, ethyl ethacrylate, n-butyl ethacrylate, isobutyl ethacrylate, t-butyl ethacrylate, 2-ethylhexyl ethacrylate, decyl ethacrylate, 2,3-dihydroxypropyl acrylate, 2,3-dihydroxypropyl methacrylate, 2-hydroxyethyl acrylate, hydroxypropyl acrylates, 2-hydroxyethyl methacrylate, 2-hydroxyethyl ethacrylate, 2-methoxyethyl acrylate, 2-methoxyethyl methacrylate, 2-methoxyethyl ethacrylate, 2-ethoxyethyl methacrylate, 2-ethoxyethyl ethacrylate, hydroxypropyl methacrylates, glyceryl monoacrylate, glyceryl monomethacrylate, polyalkylene glycol (meth)acrylates, unsaturated sulfonic acids, such as, for example, acrylamidopropanesulfonic acid;

also suitable as monomers (A-2) are maleic acid, fumaric acid, maleic anhydride and its half-esters, crotonic acid, itaconic acid.

Of these, particular preference is given to acrylic acid, methacrylic acid, maleic acid, fumaric acid, crotonic acid, maleic anhydride and half-esters thereof, methyl acrylate, methyl methacrylate, ethyl acrylate, ethyl methacrylate, n-butyl acrylate, n-butyl methacrylate, t-butyl acrylate, t-butyl methacrylate, isobutyl acrylate, isobutyl methacrylate, 2-ethylhexyl acrylate, N-t-butylacrylamide, N-octylacrylamide, 2-hydroxyethyl acrylate, hydroxypropyl acrylate, 2-hydroxyethyl methacrylate, hydroxypropyl methacrylate, alkylene glycol (meth)acrylates, unsaturated sulfonic acids, such as, for example, acrylamidopropanesulfonic acid, and N-[3-(dimethylamino)propyl]methacrylamide.

Particularly suitable monomers (A-2) are n-butyl acrylate, acrylic acid and/or stearyl methacrylate.

R₈ and R₁₂, R₁₃, may, independently of one another, be branched or unbranched C₁-C₄₀-alkyl radicals, preferably branched or unbranched C₁-C₂₀-alkyl radicals.

Suitable monomers (B-1) are monomers of the formula (III) 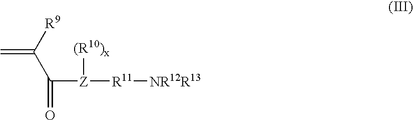
where
R⁹=H, alkyl having 1 to 8 carbon atoms,
R¹⁰=H, methyl,
R¹¹=alkylene having 1 to 24 carbon atoms, optionally substituted by C₁-C₆-alkyl,
R¹², R¹³=C₁-C₄₀-alkyl radical,
Z=nitrogen when x=1 or oxygen when x=0
x=0.

The amides may be unsubstituted, N-alkyl or N-alkylamino monosubstituted, or N,N-dialkyl-substituted or N,N-dialkylamino-disubstituted, in which the alkyl or alkylamino groups are derived from C₁-C₄₀ linear, C₃-C₄₀ branched-chain, or C₃-C₄₀ carbocyclic units. In addition, the alkylamino groups may be quaternized.

Monomers (E) which can likewise be used are substituted acrylic acids, and esters and amides thereof, where the substituents on the carbon atoms are in the two or three position of the acrylic acid, and, independently of one another, are chosen from the group consisting of C₁-C₄-alkyl, CN, COOH, particularly preferably methacrylic acid, ethacrylic acid and 3-cyanoacrylic acid. These esters and amides of these substituted acrylic acids can be chosen as described above for the esters and amides of acrylic acid.

As monomers (B-1), particular preference is given to using one or more monomers chosen from the following group: N,N-dimethylaminomethyl(meth)acrylate, N,N-diethylaminomethyl (meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate.

In a particularly preferred embodiment, the monomer (B-1) used is N,N-dimethylaminopropyl (meth)acrylate.

Suitable copolymerizable monomers (B-2) which can be used are preferably ethylenically unsaturated monomers. In this connection, it is possible to use either an individual monomer or combinations of two or more monomers. Copolymerizable means that the monomers used can be polymerized using any conventional synthetic method.

For example, these may be solution polymerization, emulsion polymerization, inverse emulsion polymerization, suspension polymerization, inverse suspension polymerization or precipitation polymerization without limiting the methods which can be used thereto. In solution polymerization, water or customary organic solvents can be used as solvents. Preparation in the melt is also possible.

Monomers which can be polymerized by a reaction initiated by free radicals are preferred. The term ethylenically unsaturated means that the monomers have at least one polymerizable carbon-carbon double bond which may be mono-, di-, tri- or tetra-substituted.

In principle, all monomers mentioned as (A-1) and (A-2) are suitable provided they are different from (B-1).

In a preferred embodiment, the copolymer (A) used is a copolymer of at least one monomer (A-1) and at least two monomers (A-2).

In a preferred embodiment, the copolymer (A) used is a copolymer of at least one monomer (A-1) and at least two monomers (A-2), where the monomers (A-2) are chosen from the group consisting of n-butyl acrylate, acrylic acid and/or stearyl methacrylate.

In a preferred embodiment, the copolymer (A) used is a copolymer in which ureidomethacrylate is chosen as monomer (A-1), and at least one monomer from the group consisting of n-butyl acrylate, acrylic acid and/or stearyl methacrylate is chosen as monomer (A-2).

In a preferred embodiment of the invention, the copolymer (A) used is a copolymer of at least one monomer (A-1) and at least 2 monomers (A-2).

In a preferred embodiment, the copolymer (A) used is a copolymer in which ureidomethacrylate is chosen as monomer (A-1), and at least two monomers from the group consisting of n-butyl acrylate, acrylic acid and/or stearyl methacrylate are chosen as monomer (A-2).

In a preferred embodiment, the copolymer (B) used is a copolymer of at least one monomer (B-1) and at least two monomers (B-2).

In a preferred embodiment, the copolymer (B) used is a copolymer of N,N-dimethylaminopropyl (meth)acrylate as monomer (B-1) and at least one further monomer (B-2).

In a preferred embodiment, the copolymer (B) used is a copolymer of N,N-dimethylaminopropyl (meth)acrylate as monomer (B-1) and at least one further monomer (B-2) chosen from the group consisting of n-butyl acrylate and ureido methacrylate.

In a preferred embodiment, the molar ratios of the copolymers (A) to (B) are chosen such that they are in the range from 1:10 to 10:1, in particular in the range from 1:5 to 5:1.

The present invention further provides copolymers (B) of
(B-1) at least one monomer of the formula (III) 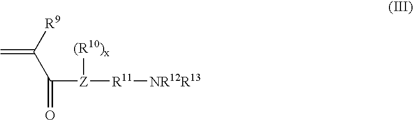
   where
   R⁹=H, alkyl having 1 to 8 carbon atoms,
   R¹⁰=H, methyl,
   R¹¹=alkylene having 1 to 24 carbon atoms, optionally substituted by C₁-C₆-alkyl,
   R¹², R¹³=C₁-C₄₀-alkyl radical,
   Z=nitrogen when x=1 or oxygen when x=0. and
      (B-2) at least one ethylenically unsaturated monomer.

The copolymers (B) are suitable for use in cosmetic preparations. The copolymers (B) are suitable as thickeners. They are suitable in particular as thickeners in cosmetic preparations.

The preparations according to the invention are suitable as thickeners. The present invention further provides a method of thickening cosmetic preparations in which 1 to 30% by weight, in particular 5 to 25% by weight, preferably 8 to 20% by weight, of a composition as claimed in claim 1 are added to the preparation to be thickened.

The preparations according to the invention can advantageously be used in cosmetic preparations, in particular hair cosmetic preparations.

The term cosmetic preparations is to be understood in a broad sense and means all those preparations which are suitable for application to skin and/or hair and/or nails and have a purpose other than a purely medicinal-therapeutic purpose.

The preparations according to the invention can be used in skin cosmetic preparations.

For example, the preparations according to the invention are used in cosmetic compositions for cleansing the skin. Such cosmetic cleansing compositions are chosen from bar soaps, such as toilet soaps, curd soaps, transparent soaps, luxury soaps, deodorant soaps, cream soaps, baby soaps, skin protection soaps, abrasive soaps and syndets, liquid soaps, such as pasty soaps, soft soaps and washing pastes, and liquid washing, showering and bath preparations, such as washing lotions, shower baths and shower gels, foam baths, oil baths and scrub preparations.

Preferably, the preparations according to the invention are used in cosmetic compositions for the care and protection of the skin, in nailcare compositions, and in preparations for decorative cosmetics.

Particular preference is given to the use in skincare compositions, intimate care compositions, foot care compositions, deodorants, light protection compositions, repellants, shaving compositions, hair-removal compositions, antiacne compositions, make-up, mascara, lipsticks, eyeshadows, kohl pencils, eyeliners, blushers, powders and eyebrow pencils.

The skincare compositions are in particular in the form of W/O or O/W skin creams, day and night creams, eye creams, face creams, antiwrinkle creams, moisturizing creams, bleaching creams, vitamin creams, skin lotions, care lotions and moisturizing lotions.

In the cosmetic preparations, the preparations according to the invention can develop particular effects. The preparations can, inter alia, contribute to the moisturization and conditioning of the skin and to the improvement in the feel of the skin. The preparations can also act as thickeners in the formulations. As a result of the addition of the preparations according to the invention, a considerable improvement in the skin compatibility can be achieved in certain formulations.

The copolymers according to the invention are present in the skin cosmetic preparations in an amount of from about 0.001 to 20% by weight, preferably 0.01 to 10% by weight, very particularly preferably 0.1 to 5% by weight, based on the total weight of the composition.

Depending on the field of use, the compositions according to the invention can be applied in a form suitable for skincare, such as, for example, in the form of a cream, foam, gel, pencil, powder, mousse, milk or lotion.

As well as the preparations according to the invention and suitable solvents, the skin cosmetic preparations can also comprise additives customary in cosmetics, such as emulsifiers, preservatives, perfume oils, cosmetic active ingredients, such as phytantriol, vitamin A, E and C, retinol, bisabolol, panthenol, light protection agents, bleaching agents, colorants, tinting agents, tanning agents (e.g. dihydroxyacetone), collagen, protein hydrolysates, stabilizers, pH regulators, dyes, salts, thickeners, gel formers, bodying agents, silicones, moisturizers, refatting agents and further customary additives.

Suitable solvents are, in particular, water and lower monoalcohols or polyols having 1 to 6 carbon atoms or mixtures thereof; preferred monoalcohols or polyols are ethanol, isopropanol, propylene glycol, glycerol and sorbitol.

Further customary additives which may be present are fatty bodies, such as mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as for example, triglycerides of C₆-C₃₀-fatty acids, wax esters, such as, for example, jojoba oil, fatty alcohols, vaseline, hydrogenated lanolin and acetylated lanolin. It is of course also possible to use mixtures thereof.

Customary thickeners in such formulations are crosslinked polyacrylic acids and derivatives thereof, polysaccharides, such as xanthan gum, agar agar, alginates or Tyloses, carboxymethylcellulose or hydroxycarboxymethylcellulose, fatty alcohols, monoglycerides and fatty acids, polyvinyl alcohol and polyvinylpyrrolidone.

The preparations according to the invention can also be mixed with conventional polymers if specific properties are to be set.

Suitable conventional polymers are, for example, anionic, cationic, amphoteric and neutral polymers.

Examples of anionic polymers are homo- and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer™ 100P), copolymers of ethyl acrylate and methacrylic acid (e.g. Luvimer™ MAE), copolymers of N-tert-butylacrylamide, ethyl acrylate, acrylic acid (Ultrahold™ 8, strong), copolymers of vinyl acetate, crotonic acid and optionally further vinyl esters (e.g. Luviset™ trade names), maleic anhydride copolymers, optionally reacted with alcohols, anionic polysiloxanes, e.g. carboxyfunctional, copolymers of vinylpyrrolidone, t-butyl acrylate, methacrylic acid (e.g. Luviskol™ VBM), copolymers of acrylic acid and methacrylic acid with hydrophobic monomers, such as, for example, C₄-C₃₀-alkyl esters of meth(acrylic acid), C₄-C₃₀-alkylvinyl esters, C₄-C₃₀-alkyl vinyl ethers and hyaluronic acid.

Further suitable polymers are cationic polymers with the INCI name Polyquaternium, e.g. copolymers of vinylpyrrolidone/N-vinylimidazolium salts (Luviquat™ FC, Luviquat™ HM, Luviquat™ MS, Luviquat™ Care), copolymers of N-vinylpyrrolidone/dimethylaminoethyl methacrylate, quaternized with diethyl sulfate (Luviquat™ PQ 11), copolymers of N-vinylcaprolactam/N-vinylpyrrolidone/N-vinylimidazolium salts (Luviquat™ Hold); cationic cellulose derivatives (Polyquaternium-4 and -10), acrylamide copolymers (Polyquaternium-7) and chitosan.

Suitable further polymers are also neutral polymers, such as polyvinylpyrrolidones, copolymers of N-vinylpyrrolidone and vinyl acetate and/or vinyl propionate, polysiloxanes, polyvinyl-caprolactam and copolymers with N-vinylpyrrolidone, polyethyleneimines and salts thereof, polyvinylamines and salts thereof, cellulose derivatives, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes or silicone resins.

The copolymers according to the invention are used in cosmetic preparations, the preparation of which takes place in accordance with the rules familiar to the person skilled in the art.

Such formulations are advantageously in the form of emulsions, preferably in the form of water-in-oil (W/O) or oil-in-water (O/W) emulsions. However, according to the invention, it is also possible and in some cases advantageous to choose other types of formulation, for example hydrodispersions, gels, oils, oleogels, multiple emulsions, for example in the form of W/O/W or O/W/O emulsion, anhydrous ointments or ointment bases, etc.

Emulsions which can be used according to the invention are prepared by known methods.

As well as the copolymers according to the invention, the emulsions comprise customary constituents, such as fatty alcohols, fatty acid esters and in particular fatty acid triglycerides, fatty acids, lanolin and derivatives thereof, natural or synthetic oils or waxes and emulsifiers in the presence of water.

The choice of additives specific to the type of emulsion and the preparation of suitable emulsions is described, for example, in Schrader, Grundlagen und Rezepturen der Kosmetika [Fundamentals and formulations of cosmetics], Hüthig Buch Verlag, Heidelberg, 2nd edition, 1989, third part, to which reference is hereby expressly made.

Thus, a skin cream which can be used according to the invention can, for example, be in the form of a W/O emulsion. An emulsion of this type comprises an aqueous phase which is emulsified by means of a suitable emulsifier system in an oil or fatty phase.

The concentration of the emulsifier system in this type of emulsion is about 4 and 35% by weight, based on the total weight of the emulsion; the fatty phase constitutes about 20 and 60% by weight and the aqueous phases about 20 and 70% by weight, in each case based on the total weight of the emulsion. The emulsifiers are those which are customarily used in this type of emulsion. They are chosen, for example, from: C₁₂C₁₈-sorbitan fatty acid esters; esters of hydroxy stearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylenated C₁₂-C₂₀-fatty alcohols; polycyclic alcohols, such as sterols; aliphatic alcohols with a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium lanolate, calcium lanolate, lithium lanolate, zinc lanolate or aluminum lanolate and hydrogenated lanolin or lanolin alcohol.

Suitable fatty components which may be present in the fatty phase of the emulsions include hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karité oil, hoplostethus oil; mineral oils, the distillation start point of which under atmospheric pressure is about 250° C. and the distillation end point of which is 410° C., such as, for example, vaseline oil; esters of saturated or unsaturated fatty acids, such as alkyl myristates, e.g. isopropyl myristate, butyl myristate or cetyl myristate, hexadecyl stearate, ethyl or isopropyl palmitate, octanoic or decanoic triglycerides and cetyl ricinoleate.

The fatty phase can also comprise silicone oils soluble in other oils, such as dimethylpolysiloxane, methylphenylpolysiloxane and the silicone glycol copolymer, fatty acids and fatty alcohols.

In order to favor the retention of oils, it is also possible to use waxes, such as, for example, carnauba wax, candelilla wax, beeswax, microcrystalline wax, ozokerite wax and Ca, Mg and Al oleates, myristates, linoleates and stearates.

In general, these water-in-oil emulsions are prepared by adding the fatty phase and the emulsifier to the batch container. The latter is heated at a temperature of from 70 to 75° C., then the ingredients soluble in oil are added and, with stirring, water which has been heated beforehand to the same temperature and in which the water-soluble ingredients have been dissolved beforehand is added; the mixture is stirred until an emulsion of the desired fineness is reached, then it is left to cool to room temperature, with less stirring if appropriate.

In addition, a care emulsion according to the invention can be in the form of a O/W emulsion. Such an emulsion usually comprises an oil phase, emulsifiers which stabilize the oil phase in the water phase, and an aqueous phase which is usually present in thickened form.

The aqueous phase of the O/W emulsion of the preparations according to the invention optionally comprises
alcohols, diols or polyols, and ethers thereof, preferably ethanol, isopropanol, propylene glycol, glycerol, ethylene glycol monoethyl ether;
customary thickeners or gel formers, such as, for example, crosslinked polyacrylic acids and derivatives thereof, polysaccharides, such as xanthan gum or alginates, carboxymethylcellulose or hydroxycarboxymethylcellulose, fatty alcohols, polyvinyl alcohol and polyvinylpyrrolidone.

The oil phase comprises oil components customary in cosmetics, such as, for example:
esters of saturated and/or unsaturated, branched and/or unbranched C₃-C₃₀-alkanecarboxylic acids and saturated and/or unsaturated, branched and/or unbranched C₃-C₃₀-alcohols, of aromatic carboxylic acids and saturated and/or unsaturated, branched and/or unbranched C₃-C₃₀-alcohols, for example isopropyl myristate, isopropyl stearate, hexyldecyl stearate, oleyl oleate; also synthetic, semisynthetic and natural mixtures of such esters, such as jojoba oil;
branched and/or unbranched hydrocarbons and hydrocarbon waxes;
silicone oils, such as cyclomethicone, dimethylpolysiloxane, diethylpolysiloxane, octamethylcyclotetrasiloxane, and mixtures thereof;
dialkyl ethers;
mineral oils and mineral waxes;
triglycerides of saturated and/or unsaturated, branched and/or unbranched C₈-C₂₄-alkanecarboxylic acids; they can be chosen from synthetic, semisynthetic or natural oils, such as olive oil, palm oil, almond oil or mixtures.

Suitable emulsifiers are preferably O/W emulsifiers, such as polyglycerol esters, sorbitan esters or partially esterified glycerides.

The preparation can take place by melting the oil phase at about 80° C.; the water-soluble constitutents are dissolved in hot water, added to the oil phase slowly and with stirring; homogenized and stirred until cold.

The preparations according to the invention are also suitable for use in washing and shower gel formulations and also bathing preparations.

As well as the preparations according to the invention, such formulations usually also comprise anionic surfactants as base surfactants and amphoteric and nonionic surfactants as cosurfactants, and also lipids, perfume oils, dyes, organic acids, preservatives and antioxidants, and also thickeners/gel formers, skin conditioning agents and moisturizers.

In the washing, showering and bathing preparations, all anionic, neutral, amphoteric or cationic surfactants used customarily in body-cleansing compositions may be used.

The formulations comprise 2 to 50% by weight of surfactants, preferably 5 to 40% by weight, particularly preferably 8 to 30% by weight.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable are, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, ethoxylated fatty acid amides, alkylpolyglycosides or sorbitan ether ester.

In addition, the washing, showering and bathing preparations can comprise customary cationic surfactants, such as, for example, quaternary ammonium compounds, for example cetyltrimethylammonium chloride.

In addition, further customary cationic polymers can also be used, thus, for example, copolymers of acrylamide and dimethyldiallylammonium chloride (Polyquaternium-7), cationic cellulose derivatives (Polyquaternium-4, -10), guar hydroxypropyltrimethylammonium chloride (INCI: Hydroxypropyl Guar Hydroxypropyltrimonium Chloride), copolymers of N-vinylpyrrolidone and quaternized N-vinylimidazole (Polyquaternium-16, -44, -46), copolymers of N-vinypyrrolidone/dimethylaminoethyl methacrylate, quaternized with diethyl sulfate (Polyquaternium-11) and others.

In addition, the washing and shower gel formulations and bathing preparations can comprise thickeners, such as, for example sodium chloride, PEG-55, propylene glycol oleate, PEG-120 methyl glucose dioleate and others, and also preservatives, further active ingredients and auxiliaries and water.

Hair cosmetic preparations include in particular styling compositions and/or conditioning compositions in hair cosmetic preparations such as hair cures, hair mousses, (hair) gels or hair sprays, hair lotions, hair rinses, hair shampoos, hair emulsions, split-end fluids, neutralizing compositions for permanent waves, hair colorants and bleaches, “hot-oil treatment” preparations, conditioners, setting lotions or hair sprays. Depending on the field of use, the hair cosmetic preparations can be applied as (aerosol) spray, (aerosol) mousse, gel, gel spray, cream, lotion or wax.

The hair cosmetic formulations according to the invention comprise, in a preferred embodiment,

a) 0.05 bis 20% by weight of the preparation according to the invention

b) 20 to 99.95% by weight of water and/or alcohol

c) 0 to 79.5% by weight of further constituents

Alcohol is understood as meaning all alcohols customary in cosmetics, e.g. ethanol, isopropanol, n-propanol.

Further constituents are understood as meaning additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active ingredients, UV filters, care substances such as panthenol, collagen, vitamins, protein hydrolysates, alpha- and beta-hydroxycarboxylic acids, protein hydrolysates, stabilizers, pH regulators, dyes, viscosity regulators, gel formers, dyes, salts, humectants, refatting agents, complexing agents and other customary additives.

Also included here are all styling and conditioning polymers known in cosmetics which can be used in combination with the polymers according to the invention if very specific properties are to be set.

Examples of suitable conventional hair cosmetic polymers are anionic polymers. Such anionic polymers are homo- and copolymers of acrylic acid and methacrylic acid or salts thereof, copolymers of acrylic acid and acrylamide and salts thereof; sodium salts of polyhydroxy-carboxylic acids, water-soluble or water-dispersible polyesters, polyurethanes (Luviset™ P.U.R.) and polyureas. Particularly suitable polymers are copolymers of t-butyl acrylate, ethyl acrylate, methacrylic acid (e.g. Luvimer™ 100P), copolymers of N-tert-butylacrylamide, ethyl acrylate, acrylic acid (Ultrahold™ 8, Strong), copolymers of vinyl acetate, crotonic acid and optionally further vinyl esters (e.g. Luviset™ trade names), maleic anhydride copolymers, optionally reacted with alcohols, anionic polysiloxanes, e.g. carboxyfunctional, copolymers of vinylpyrrolidone, t-butyl acrylate, methacrylic acid (e.g. Luviskol™ VBM).

In addition, the group of polymers suitable for combining with the polymers according to the invention includes, by way of example, Balancer CR (National Starch; acrylate copolymer), Balancer 0/55 (National Starch; acrylate copolymer), Balancer 47 (National Starch; octylacrylamide/acrylate/butylaminoethyl methacrylates copolymer), Aquaflex® FX 64 (ISP; isobutylene/ethylmaleimide/hydroxyethylmaleimide copolymer), Aquaflex®D SF-40 (ISP/National Starch; VP/vinyl caprolactam/DMAPA acrylate copolymer), Allianz® LT-120 (ISP/Rohm & Haas; acrylate/C1-2 succinate/hydroxy acrylate copolymer), Aquarez® HS (Eastman; Polyester-1), Diaformer® Z-400 (Clariant; methacryloylethylbetaine/methacrylate copolymer), Diaformer® Z-711 (Clariant; methacryloylethyl N-oxide/methacrylate copolymer), Diaformer® Z-712 (Clariant; methacryloylethyl N-oxide/methacrylate copolymer), Omnirez® 2000 (ISP; monoethyl ester of poly(methyl vinyl ether/maleic acid in ethanol), Amphomer® HC (National Starch; acrylate/octylacrylamide copolymer), Amphomer® 28-4910 (National Starch; octylacrylamide/acrylate/butylaminoethyl methacrylate copolymer), Advantage® HC 37 (ISP; terpolymer of vinylcaprolactam/vinylpyrrolidone/dimethylaminoethyl methacrylate), Acudyner 258 (Rohm & Haas; acrylate/hydroxy ester acrylate copolymer), Luviset® PUR (BASF, Polyurethane-1), Luviflex® Silk (BASF), Eastman® AQ48 (Eastman).

Very particularly preferred anionic polymers are acrylates with an acid number greater than or equal to 120 and copolymers of t-butyl acrylate, ethyl acrylate, methacrylic acid.

Further suitable hair cosmetic polymers are cationic polymers with the INCI name Polyquaternium, e.g. copolymers of vinylpyrrolidone/N-vinylimidazolium salts (Luviquat® FC, Luviquat®D HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam N-vinylpyrrolidone/N-vinylimidazolium salts (Luviquat® Hold); cationic cellulose derivatives (Polyquaternium-4 and -10), acrylamide copolymers (Polyquaternium-7).

In addition, it is also possible to use cationic guar derivatives, such as guar hydroxypropyltrimonium chloride (INCI).

Further suitable hair cosmetic polymers are also neutral polymers, such as polyvinylpyrrolidones, copolymers of N-vinylpyrrolidone and vinyl acetate and/or vinyl propionate, polysiloxanes, polyvinylcaprolactam and copolymers with N-vinylpyrrolidone, polyethyleneimines and salts thereof, polyvinylamines and salts thereof, cellulose derivatives, polyaspartic acid salts and derivatives.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyether siloxanes, silicone resins or dimethicone copolyols (CTFA) and amino functional silicone compounds such as amodimethicones (CTFA).

The preparations according to the invention are particularly suitable as setting agents in hair styling preparations, in particular hair sprays (aerosol sprays and pump sprays without propellant gas) and hair mousses (aerosol mousses and pump mousses without propellant gas).

In a preferred embodiment, these preparations comprise

a) 0.1 to 10% by weight of the preparation according to the invention

b) 20 to 99.9% by weight of water and/or alcohol

c) 0 to 70% by weight of a propellant

d) 0 to 20% by weight of further constituents

Propellants are the propellants customarily used for hair sprays or aerosol mousses. Preference is given to mixtures of propane/butane, pentane, dimethyl ether, 1,1-difluoroethane (HFC-152 a), carbon dioxide, nitrogen or compressed air.

A formulation for aerosol hair mousses which is preferred according to the invention comprises

a) 0.1 to 10% by weight of the preparation according to the invention

b) 55 to 99.8% by weight of water and/or alcohol

c) 5 to 20% by weight of a propellant

d) 0.1 to 5% by weight of an emulsifier

e) 0 to 10% by weight of further constituents

As emulsifiers, it is possible to use all emulsifiers used customarily in hair mousses. Suitable emulsifiers may be nonionic, cationic or anionic or amphoteric.

Examples of nonionic emulsifiers (INCI nomenclature) are laureths, e.g. Laureth-4; ceteths, e.g. Ceteth-1, polyethylene glycol cetyl ether; ceteareths, e.g. Ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methyl sulfate, Quaternium-1 to x (INCI).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acylisethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels may, for example, have the following composition:

a) 0.1 to 10% by weight of the preparation according to the invention

b) 60 to 99.85% by weight of water and/or alcohol

c) 0.05 to 10% by weight of a gel former

d) 0 to 20% by weight of further constituents

Gel formers which can be used are all gel formers customary in cosmetics. These include slightly crosslinked polyacrylic acid, for example Carbomer (INCI), cellulose derivatives, e.g. hydroxypropylcellulose, hydroxyethylcellulose, cationically modified celluloses, polysaccharides, e.g. Xanthum Gum, Caprylic/Capric Triglyceride, Sodium acrylates Copolymer, Polyquaternium-32 (and) Paraffinum Liquidum (INCI), Sodium Acrylates Copolymer (and) Paraffinum Liquidum (and) PPG-1 Trideceth-6, Acrylamidopropyl Trimonium Chloride/Acrylamide Copolymer, Steareth-10 Allyl Ether Acrylates Copolymer, Polyquaternium-37 (and) Paraffinum Liquidum (and) PPG-1 Trideceth-6, Polyquaternium 37 (and) Propylene Glycole Dicaprate Dicaprylate (and) PPG-1 Trideceth-6, Polyquaternium-7, Polyquaternium-44.

The preparations according to the invention increase the combability of hair, they can therefore be used as conditioning agents, in particular as conditioning agents in cosmetic preparations.

The preparations according to the invention can be used as thickeners in cosmetic preparations.

The polymers according to the invention can also be used in shampoo formulations as setting agents and/or conditioning agents. Suitable conditioning agents are in particular polymers with a cationic charge. Preferred shampoo formulations comprise

a) 0.05 to 10% by weight of the preparations according to the invention

b) 25 to 94.95% by weight of water

c) 5-50% by weight of surfactants

c) 0-5% by weight of a further conditioning agent

d) 0-10% by weight of further cosmetic constituents

In the shampoo formulations, it is possible to use all anionic, neutral, amphoteric or cationic surfactants used customarily in shampoos.

Suitable anionic surfactants are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

Suitable are, for example, sodium lauryl sulfate, ammonium lauryl sulfate, sodium lauryl ether sulfate, ammonium lauryl ether sulfate, sodium lauroyl sarcosinate, sodium oleyl succinate, ammonium lauryl sulfosuccinate, sodium dodecylbenzenesulfonate, triethanolamine dodecyl-benzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or amphopropionates, alkyl amphodiacetates or amphodipropionates.

For example, it is possible to use cocodimethylsulfopropylbetaine, laurylbetaine, cocamidopropylbetaine or sodium cocamphopropionate.

Examples of suitable nonionic surfactants are the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. Also suitable are alkylamine oxides, mono- or dialkylalkanolamides, fatty acid esters of polyethylene glycols, alkyl polyglycosides or sorbitan ether esters.

Furthermore, the shampoo formulations may comprise customary cationic surfactants, such as, for example, quaternary ammonium compounds, for example cetyltrimethylammonium chloride.

In the shampoo formulations, to achieve certain effects, customary conditioning agents can be used in combination with the polymers according to the invention. These include, for example, cationic polymers with the INCI name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). It is also possible to use protein hydrolysates, and conditioning substances based on silicone compounds, for example polyalkylsiloxanes, polyarylsiloxanes, polyarylalkylsiloxanes, polyether siloxanes or silicone resins. Further suitable silicone compounds are dimethicone copolyols (CTFA) and amino functional silicone compounds such as amodimethicones (CTFA). It is also possible to use guar derivatives, such as guar hydroxypropyltrimonium chloride (INCI).

### PREPARATION EXAMPLES

All copolymers (A) and (B) were prepared via solution polymerization as follows:

A solution of the various monomers (10% of the total amount which is used) and a solution comprising 1% of WAKO V 59 in ethanol (13% of the total amount which is used) in ethanol (70 to 250 g, depending on the polymer) was heated to 80° C. under a nitrogen atmosphere. Two different solutions were added dropwise to this solution: 1) a solution with the initiator [WAKO V59 0.99 g in ethanol (100 g)]; 2) a solution with the chosen monomers in the chosen percentage amounts. The initiator solution 1) was added dropwise over a period of 4 h, the monomer solution 2) over a period of 3 h. The reaction temperature was kept at 80° C. and the solutions were kept under a nitrogen atmosphere with continuous stirring. One hour after the last addition, a solution of 2.31 g of WAKO V59 in 100 g of ethanol was added over a period of 1 h while retaining the reaction conditions. The resulting reaction mixture was left for a further 4 h at 80° C. (with continuous stirring and under a nitrogen atmosphere). Table 1 gives the various monomers and also the mol percentage of the monomers in the corresponding copolymer.

Viscosity Measurement

The viscosity was determined for each polymer in a Wells-Brookfield CONE/PLATE viscometer with Cone CP-41 (3°) at 23° C. For this, about 2 ml of the sample were placed in the thermostated measuring body (plate was completely wetted during measurement). The viscosities were determined in mPa*s.

The viscosities were measured here in each case under three different conditions:

I) in an ethanolic solution with 40% solids content

II) in a 50:50 mixture of a) and Miglyol

III) in a 20:80 mixture of a) and Miglyol (=80)

The corresponding results are given in table 1.

TABLE 1
Synthesized copolymers (A) and (B) and their viscosities

| | Monomer | Viscos- | Viscos- | Viscos- | |
|---|---|---|---|---|---|
| | composition | ity I | ity II | ity III | Solution |
| No. | (mol %) | (mPas) | (mPas) | (mPas) | III |
| | ⁿBA:DMAPMA | | | | |
| | (Copolymer B) | | | | |
| 5 | 95:5 | 37 | 31 | 25 | Clear |
| 6 | 90:10 | 46 | 39 | 28 | Clear |
| 7 | 80:20 | 62 | 59 | 38 | Clear |
| 8 | 70:30 | 74 | 47 | 33 | Clear |
| | ⁿBA:DMAPMA | | | | |
| | (Copolymer B) | | | | |
| 9 | 88:12 | 80 | 59 | 38 | Clear |
| | ⁿBA:UMA:AA | | | | |
| | (Copolymer A) | | | | |
| 10 | 80:10:10 | 122 | 105 | N. m. ^{(a)} | P.s. ^{(b)} |
| 11 | 70:10:20 | 82 | 55 | N. m. ^{(a)} | P.s. ^{(b)} |
| 12 | 60:10:30 | 237 | 138 | N. m. ^{(a)} | P.s. ^{(b)} |
| | ⁿBA:UMA:DMAPMA | | | | |
| | (Copolymer B) | | | | |
| 13 | 80:10:10 | 52 | 44 | 29 | Clear |
| 14 | 70:10:20 | 64 | 50 | 32 | Clear |
| 15 | 60:10:30 | 144 | 94 | 44 | Clear |
| | SMA:ⁿBA:UMA:AA | | | | |
| | (Copolymer A) | | | | |

^{(a)} not miscible
^{(b)} phase separation

The viscosity of Miglyol on its own is 22 mPas

All of the copolymers in table I can be prepared in solutions I and II (I=40% strength by weight ethanolic and II=50:50 solution of I and Miglyol) as a clear solution.

Miglyol 812 is known under the INCI name Caprylic/Capric Triglyceride.

Preparation of the mixtures of copolymers (A) and copolymers (B) according to the invention

The corresponding copolymers (A) and (B) were prepared as mixtures at room temperature in various weight ratios relative to one another as below. The following mixtures were prepared (weight ratios of copolymers (A) to (B) are given in table 2).

| Mixture | Copolymer (A) | Copolymer (B) |
|---|---|---|
| (1) | ⁿBA:UMA:AA | ⁿBA:DMAPMA |
| (2) | ⁿBA:UMA:AA | ⁿBA:UMA:DMAPMA |
| (3) | SMA:ⁿBA:UMA:AA | ⁿBA:UMA:DMAPMA |

Abbreviations used
nBA n-butyl acrylate
AA acrylic acid
DMAPMA N,N-dimethylaminopropyl(meth)acrylate
SMA stearyl methacrylate
UMA ureido methacrylate

Various mixtures were prepared from the copolymers (A) and (B). These were mixed with Miglyol in various weight ratios and the viscosity was measured as stated above. Table 2 summarizes the results.

TABLE 2
Mixtures of copolymer (A) ⁿBA:UMA:AA and copolymer (B) ⁿBA:DMAPMA

| | (A) | (B) | | | |
|---|---|---|---|---|---|
| | ⁿBA:UMA:AA | ⁿBA:DMAPMA | (A):(B) | [(A) − (B)]:Miglyol | Viscosity |
| No. | (% mol) | (% mol) | weight ratio | weight ratio | (mPas) |
| 19a | 80:10:10 | 90:10 | 1:0.7 | — | 2015 |
| 19b | | | | 50:50 | 1350 |
| 19c | | | | 20:80 | N.m. ^{(a)} |
| 19d | | | 1:1.02 | — | 2655 |
| 19e | | | | 50:50 | 1573 |
| 19f | | | | 20:80 | N.m. ^{(a)} |
| 20a | 70:10:20 | 80:20 | 1:0.7 | — | — |
| 20b | | | | 50:50 | N.m. ^{(a)} |
| 20c | | | | 20:80 | N.m. ^{(a)} |
| 20d | | | 1:1.10 | — | 10076 |
| 20e | | | | 50:50 | N.m. ^{(a)} |
| 20f | | | | 20:80 | N.m. ^{(a)} |
| 21a | 60:10:30 | 1:0.7 | — | | H.v. ^{(b)} |
| 21b | | | 50:50 | | N.m. ^{(a)} |
| 21c | | | 20:80 | | N.m. ^{(a)} |
| 21d | | | 1:1.19 | — | H.v. ^{(b)} |
| 21e | | | | 50:50 | N.m. ^{(a)} |
| 21f | | | | 20:80 | N.m. ^{(a)} |
| 22a | 80:10:10 | 88:12 | 1:1 | — | 1740 |
| 22b | | | | 50:50 | 832 |
| 22c | | | | 40:60 | 750 |
| 22d | | | | 30:70 | N.m. ^{(a)} |
| 22e | | | | 20:80 | N.m. ^{(a)} |
| 23a | 70:10:20 | 88:12 | 1:2 | — | 2310 |
| 23b | | | | 50:50 | 909 |
| 23c | | | | 40:60 | 639 |
| 23d | | | | 30:70 | N.m. ^{(a)} |
| 23e | | | | 20:80 | N.m. ^{(a)} |
| 24a | 60:10:30 | 88:12 | 1:3 | — | 3640 |
| 24b | | | | 50:50 | 2700 |
| 24c | | | | 40:60 | 1819 |
| 24d | | | | 30:70 | N.m. ^{(a)} |
| 24e | | | | 20:80 | N.m. ^{(a)} |

^{(a)} n.m. not measured
^{(b)} H.v. highly viscous

1. A composition comprising
(A) copolymer of
(A-1) at least one ethylenically unsaturated, free-radically copolymerizable monomer of the formula (I)
Y—NR¹—C(V)—NHR² (I),
where the substituents have the following meanings:
Y=an ethylenically unsaturated radical capable of free-radical polymerization
V=O, S or NH
R¹, R²=independently of one another H or a C₁-C₈-alkyl group, or both together a bridging C₂-C₄-alkylene group which may be substituted up to twice by a C₁-C₄-alkoxy group and/or hydroxyl group,
(A-2) at least one unsaturated monomer of the formula (II)
X—C(O)CR⁷═CHR⁶ (II),
where the substituents have the following meanings:
X is chosen from the group of radicals —OH, —OR⁸, NH₂, —NHR⁸, N(R⁸)₂;
the radicals R⁸ may be identical or different and are chosen from the group consisting of —H, C₁-C₄₀ linear- or branched-chain alkyl radicals, N,N-dimethylaminoethyl, 2-hydroxyethyl, 2-methoxyethyl, 2-ethoxyethyl, hydroxypropyl, methoxypropyl or ethoxypropyl;
R⁷ and R⁶ are independently of one another chosen from the group consisting of —H, C₁-C₈ linear- or branched-chain alkyl chains, methoxy, ethoxy, 2-hydroxyethoxy, 2-methoxyethoxy and 2-ethoxyethyl,
(B) at least one further copolymer different from (A) of
(B-1) at least one monomer of the formula (III) 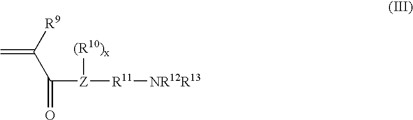
where
R⁹=H, alkyl having 1 to 8 carbon atoms,
R¹⁰=H, methyl,
R¹¹=alkylene having 1 to 24 carbon atoms, optionally substituted by C₁-C₆-alkyl,
R¹², R¹³=C₁-C₄₀-alkyl radical,
Z=nitrogen when x=1 or oxygen when x=0. and
(B-2) at least one ethylenically unsaturated monomer.

2. A composition as claimed in claim 1, wherein a copolymer of at least one monomer (A-1) and at least two monomers (A-2) is used as copolymer (A).

3. A composition as claimed in claim 1, wherein a compound of the following formula (Ia) is used as monomer (A-1) 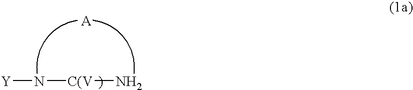
where A=a 2- or 3-membered alkylene group optionally having a carbonyl group.

4. A composition as claimed in claim 1, wherein a compound of the formula (Ib) is used as monomer (A-1)
where R³ and R⁴, independently of one another, are H, —OH, —NH, C₁-C₈-alkyl.

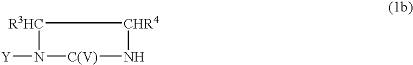

5. A composition as claimed in claim 4, where R³ and R⁴=H.

6. A composition as claimed in claim 1, wherein a compound in which V=O is used as monomer (A-1).

7. A composition as claimed in claim 1, wherein a compound of the formula (Ib) where R³ and R⁴=H and V=O, and Y═CH₂═C(CH₃)—CO—O—(CH₂)₂— is used as monomer (A-1).

8. A composition as claimed in claim 1, wherein the ethylenically unsaturated radical Y capable of free-radical polymerization is a radical of the formula (IV)
Y═CH₂═CR⁵—CO—W—(CH₂)ₙ— (IV),
where
R⁵=H, CH₃
W=O, NH
n=2 to 8, in particular 2 to 4.

9. A composition as claimed in claim 1, wherein at least one monomer which is chosen from the group consisting of N,N-dimethylaminomethyl(meth)acrylate, N,N-diethylaminomethyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate is used as monomer (B-1).

10. A composition as claimed in claim 1, wherein N,N-dimethylaminopropyl(meth)acrylate is used as monomer (B-1).

11. A composition as claimed in claim 1, wherein a copolymer of (A-1) ureidomethacrylate and (A-2) at least 2 further monomers chosen from the group consisting of n-butylacrylate, acrylic acid and stearyl methacrylate is used as copolymer (A).

12. A composition as claimed in claim 1, wherein a copolymer of (B-1) N,N-dimethylaminopropyl(meth)acrylate and at least one further monomer chosen from the group consisting of n-butyl acrylate and ureidomethacrylate is used as copolymer (B).

13. A composition as claimed in claim 1, wherein the (molar) ratio of copolymer (A) to copolymer (B) is in the range from 1:10 to 10:1, in particular in the range from 1:5 to 5:1.

14. The use of a composition as claimed in claim 1 as thickener.

15. The use of a composition as claimed in claim 1 as conditioning agent.

16. The use of a composition as claimed in claim 1 in cosmetic preparations.

17. A copolymer (B) of
(B-1) at least one monomer of the formula (III) 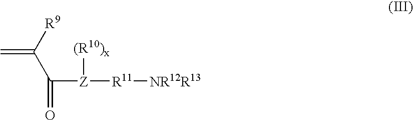
in which
R⁹=H, alkyl having 1 to 8 carbon atoms,
R¹⁰=H, methyl,
R¹¹=alkylene having 1 to 24 carbon atoms, optionally substituted by C₁-C₆-alkyl,
R¹², R¹³=C₁-C₄₀-alkyl radical,
Z=nitrogen when x=1 or oxygen when x=0. and
(B-2) at least one ethylenically unsaturated monomer.

18. The use of a copolymer (B) as claimed in claim 17 in cosmetic preparations.

19. The use of a copolymer (B) as claimed in claim 17 as thickener.

20. A method of thickening cosmetic preparations in which 1 to 30% by weight, in particular 5 to 25% by weight, preferably 8 to 20% by weight, of a composition as claimed in claim 1 are added to the preparation to be thickened.
